(19) United States
(12) Patent Application Publication
(10) Pub. No.: US 2004/0009139 A1
(43) Pub. Date: Jan. 15, 2004

(54) Nongermicidal compositions

(75) Inventor: Louis Oldenhove, Liege (BE)

Correspondence Address: PATENT DEPARTMENT, COLGATE-PALMOLIVE COMPANY, P.O. BOX 1343, PISCATAWAY, NJ, 08855-1343, US

(73) Assignee: Colgate-Palmolive Company

(21) Appl. No.: 10/350,371

(22) Filed: Jan. 23, 2003

Related U.S. Application Data
(63) Continuation-in-part of application No. 10/193,057, filed on Jul. 11, 2002.

(51) Int. Cl. C08L 3/02; C08L 5/00; A61K 7/06; A61K 31/715; A61K 31/736

(52) U.S. Cl. 424/70.13; 106/205.01; 106/124.1; 106/154.4; 106/154.11; 106/156.1; 106/205.2; 106/205.6; 106/205.7; 514/8; 514/54

(57) ABSTRACT

A nongermicidal composition which comprises an aqueous solution of an organic polymer, wherein the solution when applied to a substrate prevents the growth of microorganisms.

### RELATED APPLICATION

This application is a continuation in part application of U.S. Ser. No. 10/193,057 filed Jul. 11, 2002.

### FIELD OF THE INVENTION

A nongermicidal composition which comprises an aqueous solution of an organic polymer, wherein the solution when applied to a substrate prevents the growth of microorganisms.

### BACKGROUND OF THE INVENTION

Many commercial products claiming a disinfecting or biostatic action can be found in the market place. These products are based on various chemistries or action modes that are designed to deliver an instantaneous disinfecting or sanitizing action at the point of use. More recently, numerous products combining disinfection with a cleaning function have been commercialized in various categories, mainly household, oral care and personal care.

U.S. Pat. Nos. 5,843,861; 5,908,856 and 5,922,693 teach that it is possible to increase the disinfection potential of a peroxide bleach by incorporating hydrogen peroxide in a liquid composition lying in the vicinity of a tricritical point. The benefit of such a system resides in the in-depth penetration of the active molecules into the substrate pores and crevices.

U.S. Pat. Nos. 5,962,391 and 5,908,856 teach that the germ growth prevention effect of similar near-tricritical compositions can gain lasting efficacy by adding well chosen biostatic agents.

U.S. Pat. No. 6,132,638 relates to the prevention of dust which is possible from attaching on surfaces with appropriate surface treatments (U.S. Pat. No. 6,132,638).

### SUMMARY OF THE INVENTION

The present invention relates to a class of organic polymers which are able to prevent the growth of microorganisms by impeding adhesion of the microorganisms on substrates treated with these molecules.

The nongermicidal compositions of the present invention relates to an aqueous solution of a select class of organic polymers which can prevent the growth of microorganisms on various substrates. The aqueous solutions of the various organic polymers, can optionally include surfactants, nonaqueous solvents, cosurfactants, solubilizing agents, builder salts, sequestering agents, thickening agents, and perfumes.

### DETAILED DESCRIPTION OF THE INVENTION

The nongermicidal compositions of the instant invention comprise approximately by weight:

(a) 0.025% to 3.0% of an organic polymer selected from the group consisting of polysaccharides, xanthan gum, tragacanth gum, guar gum, iota carrageenan, karaya gum, gellan gum and the glycoprotein mucin and mixtures thereof; and

(b) the balance being water.

This nongermicidal composition can optionally contain approximately by weight:

(a) 0 to 35%, more preferably 0.5% to 32% and most preferably 1.0% to 5% of a surfactant selected from the group consisting of ethoxylated nonionic surfactants, olefin sulfonate surfactants, sulfated anionic surfactants, sulfonated anionic surfactants, an alkali metal salt of a fatty acid, an alkyl polyglucoside surfactant, a zwitterionic surfactant, an amine oxide surfactant, a lecithin, a cationic surfactant and a fatty acid alkanol amide and mixtures thereof;

(b) 0 to 15%, more preferably 0.5% to 12% of a glycol ether cosurfactant;

(c) 0 to 8%, more preferably 0.1% to 6% of a nonaqueous solvent which is an aliphatic hydrocarbon having about 6 to about 12 carbon atoms;

(d) 0 to 15%, more preferably 0.5% to 10% of at least one solubilizing agent;

(e) 0 to 25 wt. %, more preferably 0.5% to 20% of an inorganic builder salt;

(f) 0 to 15%, more preferably 0.5% to 10% of a sequestering agent;

(g) 0 to 5%, more preferably 0.1% to 3% of a polymeric thickening agent; and/or

(h) 0 to 6%, more preferably 0.05% to 3% of a perfume.

The water soluble nonionic surfactants which is utilized in this invention are commercially well known and include the primary aliphatic alcohol ethoxylates, secondary aliphatic alcohol ethoxylates, alkylphenol ethoxylates and ethylene-oxide-propylene oxide condensates on primary alkanols, such a Plurafacs (BASF) and condensates of ethylene oxide with sorbitan fatty acid esters such as the Tweens (ICI). The nonionic synthetic organic detergents generally are the condensation products of an organic aliphatic or alkyl aromatic hydrophobic compound and hydrophilic ethylene oxide groups. Practically any hydrophobic compound having a carboxy, hydroxy, amido, or amino group with a free hydrogen attached to the nitrogen can be condensed with ethylene oxide or with the polyhydration product thereof, polyethylene glycol, to form a water-soluble nonionic detergent. Further, the length of the polyethenoxy chain can be adjusted to achieve the desired balance between the hydrophobic and hydrophilic elements.

The nonionic detergent class includes the condensation products of a higher alcohol (e.g., an alkanol containing 8 to 18 carbon atoms in a straight or branched chain configuration) condensed with 5 to 30 moles of ethylene oxide, for example, lauryl or myristyl alcohol condensed with 16 moles of ethylene oxide (EO), tridecanol condensed with 6 to moles of EO, myristyl alcohol condensed with about 10 moles of EO per mole of myristyl alcohol, the condensation product of EO with a cut of coconut fatty alcohol containing a mixture of fatty alcohols with alkyl chains varying from 10 to 14 carbon atoms in length and wherein the condensate contains either 6 moles of EO per mole of total alcohol or 9 moles of EO per mole of alcohol and tallow alcohol ethoxylates containing 6 EO to 11 EO per mole of alcohol.

A preferred group of the foregoing nonionic surfactants are the Neodol ethoxylates (Shell Co.), which are higher aliphatic, primary alcohols containing about 9-15 carbon atoms, such as C₉-C₁₁ alkanol condensed with 8 moles of ethylene oxide (Neodol 91-8), C₁₂₋₁₃ alkanol condensed with 6.5 moles ethylene oxide (Neodol 23-6.5), C₁₂₋₁₅ alkanol condensed with 12 moles ethylene oxide (Neodol 25-12), C₁₄₋₁₅ alkanol condensed with 13 moles ethylene oxide (Neodol 45-13), and the like. Such ethoxamers have an HLB (hydrophobic lipophilic balance) value of 8-15 and give good/W emulsification, whereas ethoxamers with HLB values below 8 contain less than 5 ethyleneoxy groups and tend to be poor emulsifiers and poor detergents.

Additional satisfactory water soluble alcohol ethylene oxide condensates are the condensation products of a secondary aliphatic alcohol containing 8 to 18 carbon atoms in a straight or branched chain configuration condensed with 5 to 30 moles of ethylene oxide. Examples of commercially available nonionic detergents of the foregoing type are C₁₁-C₁₅ secondary alkanol condensed with either 9 EO (Tergitol 15-S-9) or 12 EO (Tergitol 15-S-12) marketed by Union Carbide.

Other suitable nonionic detergents include the polyethylene oxide condensates of one mole of alkyl phenol containing from 8 to 18 carbon atoms in a straight- or branched chain alkyl group with 5 to 30 moles of ethylene oxide. Specific examples of alkyl phenol ethoxylates include nonyl condensed with 9.5 moles of EO per mole of nonyl phenol, dinonyl phenol condensed with 12 moles of EO per mole of phenol, dinonyl phenol condensed with 15 moles of EO per mole of phenol and di-isoctylphenol condensed with 15 moles of EO per mole of phenol. Commercially available nonionic surfactants of this type include Igepal CO-630 (nonyl phenol ethoxylate) marketed by GAF Corporation.

Also among the satisfactory nonionic detergents are the water-soluble condensation products of a C₈-C₂₀ alkanol with a heteric mixture of ethylene oxide and propylene oxide wherein the weight ratio of ethylene oxide to propylene oxide is from 2.5:1 to 4:1, preferably 2.8:1-3.3:1, with the total of the ethylene oxide and propylene oxide (including the terminal ethanol or propanol group) being from 60-85%, preferably 70-80%, by weight. Such detergents are commercially available from BASF-Wyandotte and a particularly preferred detergent is a C₁₀-C₁₆ alkanol condensate with ethylene oxide and propylene oxide, the weight ratio of ethylene oxide to propylene oxide being 3:1 and the total alkoxy content being 75% by weight.

Other suitable water-soluble nonionic detergents which are less preferred are marketed under the trade name “Pluronics.” The compounds are formed by condensing ethylene oxide with a hydrophobic base formed by the condensation of propylene oxide with propylene glycol. The molecular weight of the hydrophobic portion of the molecule is of the order of 950 to 4000 and preferably 200 to 2,500. The addition of polyoxyethylene radicals to the hydrophobic portion tends to increase the solubility of the molecule as a whole so as to make the surfactant water-soluble. The molecular weight of the block polymers varies from 1,000 to 15,000 and the polyethylene oxide content may comprise 20% to 80% by weight. Preferably, these surfactants will be in liquid form and satisfactory surfactants are available as grades L62 and L64.

The anionic surfactants which may be used in the nonionic based liquid detergent of this invention are water soluble such as triethanolamine and include the sodium, potassium, ammonium and ethanolammonium salts of C₈-C₁₈ alkyl sulfates such as lauryl sulfate, myristyl sulfate and the like; linear C₈-C₁₆ alkyl benzene sulfonates; C₁₀-C₂₀ paraffin sulfonates; alpha olefin sulfonates containing about 10-24 carbon atoms; C₈-C₁₈ alkyl sulfoacetates; C₈-C₁₈ alkyl sulfosuccinate esters; C₈-C₁₈ acyl isethionates; and C₈-C₁₈ acyl taurates. Preferred anionic surfactants are the water soluble C₁₂-C₁₆ alkyl sulfates, the C₁₀-C₁₅ alkylbenzene sulfonates, the C₁₃-C₁₇ paraffin sulfonates and the alpha C₁₂-C₁₈ olefin sulfonates.

The higher alkyl mononuclear aromatic sulfonates, such as the higher alkylbenzene sulfonates containing 9 to 18 or preferably 9 to 16 carbon atoms in the higher alkyl group in a straight or branched chain. A preferred alkylbenzene sulfonate is a linear alkylbenzene sulfonate having a higher content of 3-phenyl (or higher) isomers and a correspondingly lower content (well below 50%) of 2-phenyl (or lower) isomers, such as those sulfonates wherein the benzene ring is attached mostly at the 3 or higher (for example 4, 5, 6 or 7) position of the alkyl group and the content of the isomers in which the benzene ring is attached in the 2 or 1 position is correspondingly low. Preferred materials are set forth in U.S. Pat. No. 3,320,174, especially those in which the alkyls are of 10 to 13 carbon atoms.

Examples of suitable other sulfonated anionic detergents are the well known. The paraffin sulfonates may be monosulfonates or di-sulfonates and usually are mixtures thereof, obtained by sulfonating paraffins of 10 to 20 carbon atoms. Preferred paraffin sulfonates are those of C₁₂₋₁₈ carbon atoms chains, and more preferably they are of C₁₄₋₁₇ chains. Paraffin sulfonates that have the sulfonate group(s) distributed along the paraffin chain are described in U.S. Pat. Nos. 2,503,280; 2,507,088; 3,260,744; and 3,372,188; and also in German Patent 735,096. Such compounds may be made to specifications and desirably the content of paraffin sulfonates outside the C₁₄₋₁₇ range will be minor and will be minimized, as will be any contents of di- or poly-sulfonates.

The C₈₋₁₈ ethoxylated alkyl ether sulfate surfactants have the structure

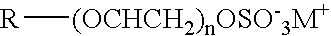

wherein n is about 1 to about 22 more preferably 1 to 3 and R is an alkyl group having about 8 to about 18 carbon atoms, more preferably 12 to 15 and natural cuts, for example, C₁₂₋₁₄ or C₁₂₋₁₆ and M is an ammonium cation or a metal cation, most preferably sodium.

The ethoxylated alkyl ether sulfate may be made by sulfating the condensation product of ethylene oxide and C₈₋₁₀ alkanol, and neutralizing the resultant product. The ethoxylated alkyl ether sulfates differ from one another in the number of carbon atoms in the alcohols and in the number of moles of ethylene oxide reacted with one mole of such alcohol. Preferred ethoxylated alkyl ether polyethenoxy sulfates contain 12 to 15 carbon atoms in the alcohols and in the alkyl groups thereof, e.g., sodium myristyl (3 EO) sulfate.

Ethoxylated C₈₋₁₈ alkylphenyl ether sulfates containing from 2 to 6 moles of ethylene oxide in the molecule are also suitable for use in the invention compositions. These detergents can be prepared by reacting an alkyl phenol with 2 to 6 moles of ethylene oxide and sulfating and neutralizing the resultant ethoxylated alkylphenol.

The C₁₂-C₂₀ paraffin sulfonates may be monosulfonates or di-sulfonates and usually are mixtures thereof, obtained by sulfonating paraffins of 10 to 20 carbon atoms. Preferred paraffin sulfonates are those of C₁₂₋₁₈ carbon atoms chains, and more preferably they are of C₁₄₋₁₇ chains. Paraffin sulfonates that have the sulfonate group(s) distributed along the paraffin chain are described in U.S. Pat. Nos. 2,503,280; 2,507,088; 3,260,744 and 3,372,188 and also in German Patent 735,096. Such compounds may be made to specifications and desirably the content of paraffin sulfonates outside the C₁₄₋₁₇ range will be minor and will be minimized, as will be any contents of di- or poly-sulfonates.

The present invention can also contain an alpha olefin sulfonates, including long-chain alkene sulfonates, long-chain hydroxyalkane sulfonates or mixtures of alkene sulfonates and hydroxyalkane sulfonates. These alpha olefin sulfonate surfactants may be prepared in a known manner by the reaction of sulfur trioxide (SO₃) with long-chain olefins containing 8 to 25, preferably 12 to 21 carbon atoms and having the formula RCH═CHR₁ where R is a higher alkyl group of 6 to 23 carbons and R₁ is an alkyl group of 1 to 17 carbons or hydrogen to form a mixture of sultones and alkene sulfonic acids which is then treated to convert the sultones to sulfonates. Preferred alpha olefin sulfonates contain from 14 to 16 carbon atoms in the R alkyl group and are obtained by sulfonating an a-olefin.

The long chain fatty acids are the higher aliphatic fatty acids having from about 8 to 22 carbon atoms, more preferably from about 10 to 20 carbon atoms, and especially preferably from about 12 to 18 carbon atoms, and especially preferably from 12 to 18 carbon atoms, inclusive of the carbon atom of the carboxyl group of the fatty acid. The aliphatic radical may be saturated or unsaturated and may be straight or branched. Straight chain saturated fatty acids are preferred. Mixtures of fatty acids may be used, such as those derived from natural sources, such as tallow fatty acid, coco fatty acid, soya fatty acid, mixtures of these acids, etc. Stearic acid and mixed fatty acids, e.g. stearic acid/palmitic acid, are preferred.

Thus, examples of the fatty acids include, for example, decanoic acid, dodecanoic acid, palmitic acid, myristic acid, stearic acid, behenic acid, oleic acid, eicosanoic acid, tallow fatty acid, coco fatty acid, soya fatty acid, mixtures of these acids, etc. Stearic acid and mixed fatty acids, e.g. stearic acid/palmitic acid, are preferred.

The water-soluble zwitterionic surfactant, which can also be used provides good foaming properties and mildness to the present nonionic based liquid detergent. The zwitterionic surfactant is a water soluble betaine having the general formula:

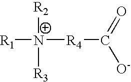

wherein R₁ is an alkyl group having 10 to 20 carbon atoms, preferably 12 to 16 carbon atoms, or the amido radical:

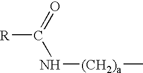

wherein R is an alkyl group having 9 to 19 carbon atoms and a is the integer 1 to 4; R₂ and R₃ are each alkyl groups having 1 to 3 carbons and preferably 1 carbon; R₄ is an alkylene or hydroxyalkylene group having from 1 to 4 carbon atoms and, optionally, one hydroxyl group. Typical alkyldimethyl betaines include decyl dimethyl betaine or 2-(N-decyl-N, N-dimethyl-ammonia) acetate, coco dimethyl betaine or 2-(N-coco N, N-dimethylammonio) acetate, myristyl dimethyl betaine, palmityl dimethyl betaine, lauryl diemethyl betaine, cetyl dimethyl betaine, stearyl dimethyl betaine, etc. The amidobetaines similarly include cocoamidoethylbetaine, cocoamidopropyl betaine and the like. A preferred betaine is coco (C₈-C₁₈) amidopropyl dimethyl betaine.

Amine oxide semi-polar nonionic surfactants comprise compounds and mixtures of compounds having the formula:

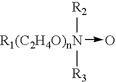

wherein R₁ is an alkyl, 2-hydroxyalkyl, 3-hydroxyalkyl, or 3-alkoxy-2-hydroxypropyl radical in which the alkyl and alkoxy, respectively, contain from 8 to 18 carbon atoms, R₂ and R₃ are each methyl, ethyl, propyl, isopropyl, 2-hydroxyethyl, 2-hydroxypropyl, or 3-hydroxypropyl, and n is from 0 to 10. Particularly preferred are amine oxides of the formula:

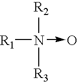

wherein R₁ is a C₁₂₋₁₆ alkyl and R₂ and R₃ are methyl or ethyl. The above ethylene oxide condensates, amides, and amine oxides are more fully described in U.S. Pat. No. 4,316,824 which is hereby incorporated herein by reference.

The instant composition can contain a mixture of a C₁₂₋₁₄ alkyl monoalkanol amide such as lauryl monoalkanol amide and a C₁₂₋₁₄ alkyl dialkanol amide such as lauryl diethanol amide or coco diethanol amide.

As used herein and in the appended claims the term “perfume” is used in its ordinary sense to refer to and include any non-water soluble fragrant substance or mixture of substances including natural (i.e., obtained by extraction of flower, herb, blossom or plant), artificial (i.e., mixture of natural oils or oil constituents) and synthetically produced substance) odoriferous substances. Typically, perfumes are complex mixtures of blends of various organic compounds such as alcohols, aldehydes, ethers, aromatic compounds and varying amounts of essential oils (e.g., terpenes) such as from 0% to 80%, usually from 10% to 70% by weight, the essential oils themselves being volatile odoriferous compounds and also serving to dissolve the other components of the perfume.

In the present invention the precise composition of the perfume is of no particular consequence to cleaning performance so long as it meets the criteria of water immiscibility and having a pleasing odor. Naturally, of course, especially for cleaning compositions intended for use in the home, the perfume, as well as all other ingredients, should be cosmetically acceptable, i.e., non-toxic, hypoallergenic, etc. The instant compositions show a marked improvement in ecotoxicity as compared to existing commercial products.

In place of the perfume one can employ an essential oil or a water insoluble hydrocarbon having 6 to 18 carbon such as a paraffin or isoparaffin.

Suitable essential oils are selected from the group consisting of: Anethole 20/21 natural, Aniseed oil china star, Aniseed oil globe brand, Balsam (Peru), Basil oil (India), Black pepper oil, Black pepper oleoresin 40/20, Bois de Rose (Brazil) FOB, Borneol Flakes (China), Camphor oil, White, Camphor powder synthetic technical, Cananga oil (Java), Cardamom oil, Cassia oil (China), Cedarwood oil (China) BP, Cinnamon bark oil, Cinnamon leaf oil, Citronella oil, Clove bud oil, Clove leaf, Coriander (Russia), Coumarin 69° C. (China), Cyclamen Aldehyde, Diphenyl oxide, Ethyl vanilin, Eucalyptol, Eucalyptus oil, Eucalyptus citriodora, Fennel oil, Geranium oil, Ginger oil, Ginger oleoresin (India), White grapefruit oil, Guaiacwood oil, Gurjun balsam, Heliotropin, Isobornyl acetate, Isolongifolene, Juniper berry oil, L-methyl acetate, Lavender oil, Lemon oil, Lemongrass oil, Lime oil distilled, Litsea Cubeba oil, Longifolene, Menthol crystals, Methyl cedryl ketone, Methyl chavicol, Methyl salicylate, Musk ambrette, Musk ketone, Musk xylol, Nutmeg oil, Orange oil, Patchouli oil, Peppermint oil, Phenyl ethyl alcohol, Pimento berry oil, Pimento leaf oil, Rosalin, Sandalwood oil, Sandenol, Sage oil, Clary sage, Sassafras oil, Spearmint oil, Spike lavender, Tagetes, Tea tree oil, Vanilin, Vetyver oil (Java), Wintergreen, Allocimene, Arbanex™, Arbanol®, Bergamot oils, Camphene, Alpha-Campholenic aldehyde, I-Carvone, Cineoles, Citral, Citronellol Terpenes, Alpha-Citronellol, Citronellyl Acetate, Citronellyl Nitrile, Para-Cymene, Dihydroanethole, Dihydrocarveol, d-Dihydrocarvone, Dihydrolinalool, Dihydromyrcene, Dihydromyrcenol, Dihydromyrcenyl Acetate, Dihydroterpineol, Dimethyloctanal, Dimethyloctanol, Dimethyloctanyl Acetate, Estragole, Ethyl-2 Methylbutyrate, Fenchol, Fernlol™, Florilys™, Geraniol, Geranyl Acetate, Geranyl Nitrile, Glidmint™ Mint oils, Glidox™, Grapefruit oils, trans-2-Hexenal, trans-2-Hexenol, cis-3-Hexenyl Isovalerate, cis-3-Hexanyl-2-methylbutyrate, Hexyl Isovalerate, Hexyl-2-methylbutyrate, Hydroxycitronellal, Ionone, Isobornyl Methylether, Linalool, Linalool Oxide, Linalyl Acetate, Menthane Hydroperoxide, I-Methyl Acetate, Methyl Hexyl Ether, Methyl-2-methylbutyrate, 2-Methylbutyl Isovalerate, Myrcene, Nerol, Neryl Acetate, 3-Octanol, 3-Octyl Acetate, Phenyl Ethyl-2-methylbutyrate, Petitgrain oil, cis-Pinane, Pinane Hydroperoxide, Pinanol, Pine Ester, Pine Needle oils, Pine oil, alpha-Pinene, beta-Pinene, alpha-Pinene Oxide, Plinol, Plinyl Acetate, Pseudo Ionone, Rhodinol, Rhodinyl Acetate, Spice oils, alpha-Terpinene, gamma-Terpinene, Terpinene-4-OL, Terpineol, Terpinolene, Terpinyl Acetate, Tetrahydrolinalool, Tetrahydrolinalyl Acetate, Tetrahydromyrcenol, Tetralol®, Tomato oils, Vitalizair, Zestoral™.

The instant compositions can contain at least one solubilizing agent selected from the group consisting of a C₂₋₅ mono, dihydroxy or polyhydroxy alkanols such as ethanol, isopropanol, glycerol ethylene glycol, diethylene glycol, propylene glycol and alkali metal cumene or xylene sulfonates such as sodium cumene sulfonate and sodium xylene sulfonate and mixtures thereof. The solubilizing agents are included in order to control low temperature cloud clear properties and aide in cleaning.

The cosurfactants used in the instant inventions are glycerol, ethylene glycol, water-soluble polyethylene glycols having a molecular weight of 300 to 1000, polypropylene glycol of the formula HO(CH₃CHCH₂O)ₙH wherein n is a number from 2 to 18, mixtures of polyethylene glycol and polypropyl glycol (Synalox) and mono C₁-C₆ alkyl ethers of ethylene glycol and propylene glycol having the structural formula R(X)ₙOH wherein R is C₁-C₆ alkyl group, X is (OCH₂CH₂) or (OCH₂(CH₃)CH) and n is a number from 1 to 4, diethylene glycol, triethylene glycol, 1methoxy-2-propanol, 1methoxy-3-propanol, and 1methoxy 2-, 3- or 4-butanol, and triethyl phosphate. Additionally, mixtures of two or more of the three classes of cosurfactant compounds may be employed where specific pH's are desired.

Representative members of the polypropylene glycol include dipropylene glycol and polypropylene glycol having a molecular weight of 200 to 1000, e.g., polypropylene glycol 400. Other satisfactory glycol ethers are ethylene glycol monobutyl ether (butyl cellosolve), diethylene glycol monobutyl ether (butyl carbitol), triethylene glycol monobutyl ether, mono, di, tri propylene glycol monobutyl ether, tetraethylene glycol monobutyl ether, mono, di, tripropylene glycol monomethyl ether, propylene glycol monomethyl ether, ethylene glycol monohexyl ether, diethylene glycol monohexyl ether, propylene glycol tertiary butyl ether, ethylene glycol monoethyl ether, ethylene glycol monomethyl ether, ethylene glycol monopropyl ether, ethylene glycol monopentyl ether, diethylene glycol monomethyl ether, diethylene glycol monoethyl ether, diethylene glycol monopropyl ether, diethylene glycol monopentyl ether, triethylene glycol monomethyl ether, triethylene glycol monoethyl ether, triethylene glycol monopropyl ether, triethylene glycol monopentyl ether, triethylene glycol monohexyl ether, mono, di, tripropylene glycol monoethyl ether, mono, di tripropylene glycol monopropyl ether, mono, di, tripropylene glycol monopentyl ether, mono, di, tripropylene glycol monohexyl ether, mono, di, tributylene glycol mono methyl ether, mono, di, tributylene glycol monoethyl ether, mono, di, tributylene glycol monopropyl ether, mono, di, tributylene glycol monobutyl ether, mono, di, tributylene glycol monopentyl ether and mono, di, tributylene glycol monohexyl ether, ethylene glycol phenyl ether and 1-phenoxy-2-propanol, ethylene glycol monoacetate and dipropylene glycol propionate.

One of the thickeners which is used in the instant invention is selected from the group consisting of hydrophobically modified polymers like Carbopol 614 manufactured by B. F. Goodrich Co. and Polygel K200, xanthan gum and Laponite caly manufactured by Southern Clay. One of the preferred thickeners, a xanthan gum called Keltrol T and sold by Merck & Co. The xanthan gum is an exocellular hetropolysaccharide having a molecular weight of about 1,000,000 to 10,000,000 and is used in a concentration of 0.1 weight percent to about 1.0 weight percent, more preferably about 0.1 to about 0.7 weight percent, and most preferably 0.2 to 0.6 weight percent. When used at these concentration levels, the composition retains its microemulsion characteristics in that the essential micellar aggregates are maintained. The composition is still sprayable and will nicely cling to a vertical wall. Additionally, the compositions having the xanthan gum incorporates therein are shear thinning which means that the composition can be easily removed from the surface being cleaned without much mechanical action.

Another thickener is a noncrosslinked quaternary acrylic acid homopolymer having a molecular weight of about 30,000 to about 2,000,000 such as Polygel K200 manufactured by 3V Inc. of Georgetown, S.C. The Polygel K200 is N,N,N,-Trimethyl-2[Methyl-1-OXO-Propenyl)Oxy]-chloride homopolymer.

Other thickeners are carrageenan, cellulose gum (KMO), hydroxyethyl cellulose (HEC), methyl cellulose, hydropropyl methyl cellulose, polyacrylamides, polyvinyl alcohol, alumina, polygenic silicas and anionic polymers.

A preferred solid inorganic builder salt is an alkali metal polyphosphate such as sodium tripolyphosphate (“TPP”). In place of all or part of the alkali metal polyphosphate one or more other detergent builder salts can be used. Suitable other builder salts are alkali metal carbonates, citrates, tartarates, borates, phosphates, bicarbonates, lower polycarboxylic acid salts, and polyacrylates, polymaleic anhydrides and copolymers of polyacrylates and polymaleic anhydrides and polyacetal carboxylates.

Specific examples of such builders are sodium carbonate, potassium carbonate, sodium citrate, potassium citrate, sodium tetraborate, sodium pyrophosphate, sodium tripolyphosphate, potassium tripolyphosphate, potassium pyrophosphate, sodium bicarbonate, sodium hexametaphosphate, sodium sesquicarbonate, sodium mono and diorthophosphate, and potassium bicarbonate. The builder salts can be used alone or in an admixture with other builders. Typical builders also include those disclosed in U.S. Pat. Nos. 4,316,812, 4,264,466 and 3,630,929 and those disclosed in U.S. Pat. Nos. 4,144,226, 4,135,092 and 4,146,495.

A preferred builder salt is sodium tripolyphosphate (TPP). The TPP is a blend of anhydrous TPP and a small amount of TPP hexahydrate such that the chemically bound water content corresponds to one H₂O per pentasodium tripolyphosphate molecule. Such TPP may be produced by treating anhydrous TPP with a limited amount of water. The presence of the hexahydrate slows down the rapid rate of solution of the TPP in the wash bath and inhibits caking. One suitable TPP is sold under the name Thermphos NW. The particles size of the Thermphos NW TPP, as supplied, is usually averages 200 microns with the largest particles being 400 microns. Potassium tripolyphosphate and potassium pyrosphosphate can also be used. Nonphosphate builders such as alkali metal carbonates, alkali metal tartartes, alkali metal gluconates and alkali metal carbonates, alkali metal citrates and mixtures thereof can be used with the phosphate builders.

The alkyl polysaccharides surfactants, which can be used have a hydrophobic group containing from about 8 to about 20 carbon atoms, preferably from about 10 to about 16 carbon atoms, most preferably from about 12 to about 14 carbon atoms, and polysaccharide hydrophilic group containing from about 1.5 to about 10, preferably from about 1.5 to about 4, most preferably from about 1.6 to about 2.7 saccharide units (e.g., galactoside, glucoside, fructoside, glucosyl, fructosyl; and/or galactosyl units). Mixtures of saccharide moieties may be used in the alkyl polysaccharide surfactants. The number x indicates the number of saccharide units in a particular alkyl polysaccharide surfactant. For a particular alkyl polysaccharide molecule x can only assume integral values. In any physical sample of alkyl polysaccharide surfactants there will be in general molecules having different x values. The physical sample can be characterized by the average value of x and this average value can assume non-integral values. In this specification the values of x are to be understood to be average values. The hydrophobic group (R) can be attached at the 2-, 3-, or 4-positions rather than at the 1-position, (thus giving e.g. a glucosyl or galactosyl as opposed to a glucoside or galactoside). However, attachment through the 1-position, i.e., glucosides, galactoside, fructosides, etc., is preferred. In the preferred product the additional saccharide units are predominately attached to the previous saccharide unit's 2-position. Attachment through the 3-, 4-, and 6-positions can also occur. Optionally and less desirably there can be a polyalkoxide chain joining the hydrophobic moiety (R) and the polysaccharide chain. The preferred alkoxide moiety is ethoxide.

Typical hydrophobic groups include alkyl groups, either saturated or unsaturated, branched or unbranched containing from about 8 to about 20, preferably from about 10 to about 18 carbon atoms. Preferably, the alkyl group is a straight chain saturated alkyl group. The alkyl group can contain up to 3 hydroxy groups and/or the polyalkoxide chain can contain up to about 30, preferably less than about 10, alkoxide moieties.

Suitable alkyl polysaccharides are decyl, dodecyl, tetradecyl, pentadecyl, hexadecyl, and octadecyl, di-, tri-, tetra-, penta-, and hexaglucosides, galactosides, lactosides, fructosides, fructosyls, lactosyls, glucosyls and/or galactosyls and mixtures thereof.

The alkyl monosaccharides are relatively less soluble in water than the higher alkyl polysaccharides. When used in admixture with alkyl polysaccharides, the alkyl monosaccharides are solubilized to some extent. The use of alkyl monosaccharides in admixture with alkyl polysaccharides is a preferred mode of carrying out the invention. Suitable mixtures include coconut alkyl, di-, tri-, tetra-, and pentaglucosides and tallow alkyl tetra-, penta-, and hexaglucosides.

The preferred alkyl polysaccharides are alkyl polyglucosides having the formula

R₂O(CₙH₂ₙO)r(Z)ₓ

wherein Z is derived from glucose, R is a hydrophobic group selected from the group consisting of alkyl, alkylphenyl, hydroxyalkylphenyl, and mixtures thereof in which said alkyl groups contain from about 10 to about 18, preferably from about 12 to about 14 carbon atoms; n is 2 or 3 preferably 2, r is from 0 to 10, preferable 0; and x is from 1.5 to 8, preferably from 1.5 to 4, most preferably from 1.6 to 2.7. To prepare these compounds a long chain alcohol (R₂OH) can be reacted with glucose, in the presence of an acid catalyst to form the desired glucoside. Alternatively the alkyl polyglucosides can be prepared by a two step procedure in which a short chain alcohol (R₁OH) can be reacted with glucose, in the presence of an acid catalyst to form the desired glucoside. Alternatively the alkyl polyglucosides can be prepared by a two step procedure in which a short chain alcohol (C₁₋₆) is reacted with glucose or a polyglucoside (x=2 to 4) to yield a short chain alkyl glucoside (x=1 to 4) which can in turn be reacted with a longer chain alcohol (R₂OH) to displace the short chain alcohol and obtain the desired alkyl polyglucoside. If this two step procedure is used, the short chain alkylglucosde content of the final alkyl polyglucoside material should be less than 50%, preferably less than 10%, more preferably less than about 5%, most preferably 0% of the alkyl polyglucoside.

The amount of unreacted alcohol (the free fatty alcohol content) in the desired alkyl polysaccharide surfactant is preferably less than about 2%, more preferably less than about 0.5% by weight of the total of the alkyl polysaccharide. For some uses it is desirable to have the alkyl monosaccharide content less than about 10%.

The used herein, “alkyl polysaccharide surfactant” is intended to represent both the preferred glucose and galactose derived surfactants and the less preferred alkyl polysaccharide surfactants. Throughout this specification, “alkyl polyglucoside” is used to include alkyl polyglycosides because the stereochemistry of the saccharide moiety is changed during the preparation reaction.

An especially preferred APG glycoside surfactant is APG 625 glycoside manufactured by the Henkel Corporation of Ambler, Pa. APG25 is a nonionic alkyl polyglycoside characterized by the formula:

CₙH₂ₙ₊₁O(C₆H₁₀O₅)ₓH

wherein n=10 (2%); n=122 (65%); n=14 (21-28%); n=16 (4-8%) and n=18 (0.5%) and x (degree of polymerization)=1.6. APG 625 has: a pH of 6 to 10 (10% of APG 625 in distilled water); a specific gravity at 25° C. of 1.1 g/ml; a density at 25° C. of 9.1 lbs/gallon; a calculated HLB of 12.1 and a Brookfield viscosity at 35C, 21 spindle, 5-10 RPM of 3,000 to 7,000 cps.

A preferred polysaccharide is Fucogel® 1000 which is available from Solabia and is a polysaccharide with the following sequence: L-fucose, D-galactose and galacturonic acid having a molecular weight of 100 to 200,000 daltons. Fucogel® is depicted by the structure of:

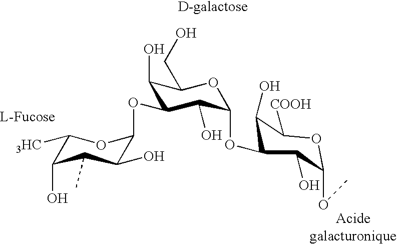

Xanthan gum has a primary structure consisting of regular repeating units, each containing five sugars: two glucose, two mannose, and one glucuronic acid. The main chain is built up of β-D-glucose units linked through the 1- and 4-positions, i.e, a chemical structure same as cellulose. A three-sugar side chain is linked to the 3-position of every other glucose residue in the main chain. About half of the terminal D-mannose residues contain a pyruvic acid residue linked to the 4- and 6-positions.

The molecular weight is several million Da.

Carrageenan gum has a galactose backbone.

There are three types of carrageenan (Kappa, Iota and Lambda) whose structures are gels with potassium (Kappa):

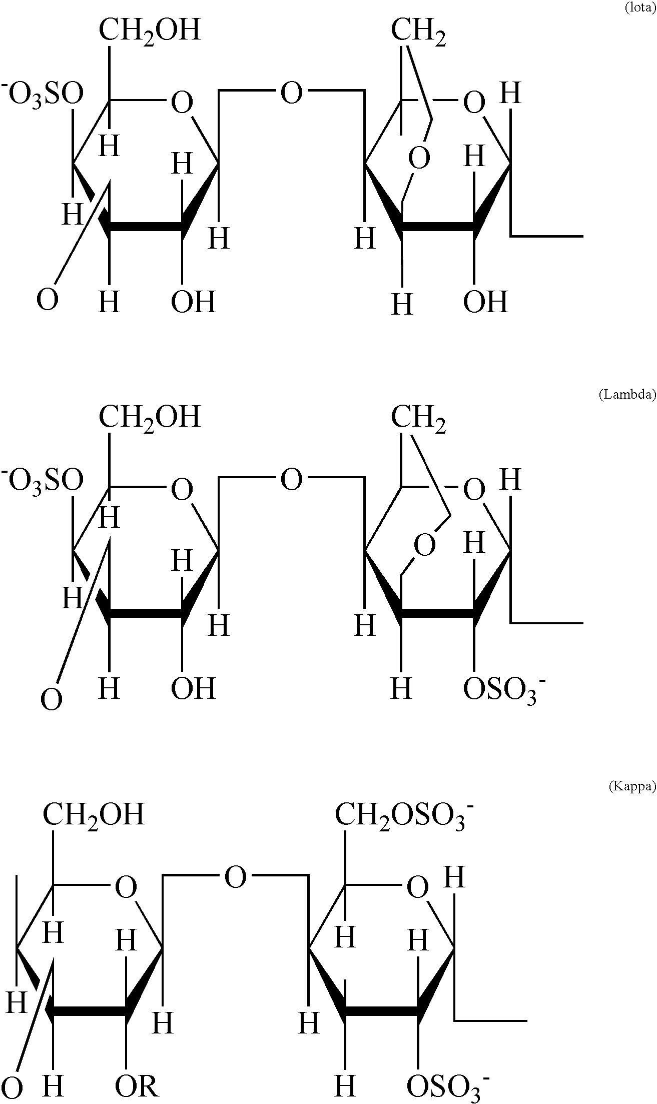

Tragacanth gum available from Sigma is a polymer of galacturonic acid and galactose and arabinose and xylose. It is made from two fractions: hydrosoluble one contains tragacanthin which is a complex mixture of polysaccharides containing D-galacturonic acid and other sugars and traces of starch and cellulose; and water insoluble fraction swells to a gel and consists of 6-70% of bassorin.

Karaya gum is available from Sigma and is acetylated galacturonic acid and rhamnose and galactose. It has a molecular weight of 800,000 to 1,100,000.

Mucin is available from Sigma and is group of large glycoproteins, major constituents of saliva, gastric juice, intestinal juice, and other secretions. It consists of a tread-like single polypeptide backbone which contains segments with densely packed carbohydrate side-chains. Carbohydrate accounts for about 50% and the protein for about 30% of the MW. Carbohydrate moieties consist in O-linked oligosaccharides which attach to the peptide backbone via its serine and threonine residues. The number of sugar residues per oligosaccharide side chain varies from 1-20; their composition is mainly the GalNAc (N-acetylgalactosamine), GlcNAc (N-acetylglucosamine), galactose, fucose and sialic acid. The structure of polygalacturonic acid is:

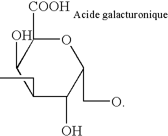

Gellan (gelrite) gum is available from Pseudomonas and is composed of 2β-Glucose units and β-Glucuronic acid and rhamnose and has a molecular weight of 500,000 to 1,100,000. The structure of gellan is:

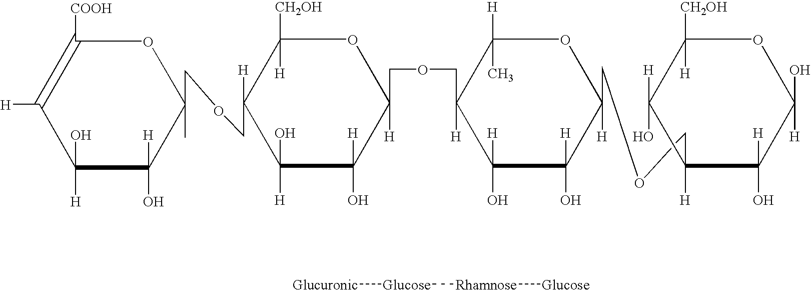

Guar gum is available from Sigma and is a galactomannan (mannose (1-4) and galactose (1-6) every other mannose and has a molecular weight of 200,000 to 240,000. It is depicted by the structure of:

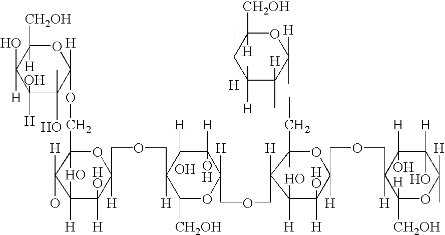

The following examples in wt. % were prepared by simple mixing.

### EXAMPLE 1

To assess the impact of surface treatment on bacteria adhesion, various coatings have been tested for anti-germ adhesion properties. The germ anti-adhesion potential of several treatments has been assessed with the following procedure.

The test is carried out with using small tiles of the substrates on which the germ anti-adhesion effects are evaluated (i.e. ceramic tiles 2.5×2.5 cm² and/or marble tiles 2.0×2.0 cm² and/or stainless steel 316L plates 2.5×2.5 cm²).

Before to be used in a test, tiles of one (or several) selected substrate(s) are cleaned with ethanol and abundantly rinsed with deionized water. They are then let dry at room temperature and sterilized during 15 minutes at 121° C. in a glass jar.

200 μl of the solutions containing the molecules (generally 0.1%) or products to be tested are laid on the sterile tiles at room temperature. Two replicates are considered per treatment.

The tiles are let dry overnight at room temperature in semi-open Petri dishes (to avoid contamination from ambient air).

To assess the resistance of the treatment coating to a water rinsing effect, a series of tiles can be reserved for this purpose and included in parallel in the test design. The next day, the tiles selected to assess rinse effects are gently rinsed with 5×10 ml sterile water. From this stage, a minimum 2h 30 time delay is needed to allow rinsed tiles to dry before starting to the inoculation step.

Germs are collected from the hands of selected volunteers just before the inoculation step: for this purpose, three volunteers are asked to, consecutively three times, take a little tap water in their hands and rub them above a 2 liters glass beaker; the resulting collected solution contains a high amount of germs.

Each tile is then inoculated with 200 μl of the above aqueous suspension of wild germs previously collected from the hands of volunteers. The ceramic tiles are laid in Petri dishes on blotting paper impregnated with 2 ml sterile deionized water to avoid the drying of germs on the surface. After a determined contact time (i.e. 2h30), the tiles are rinsed with 2×10 ml sterile tap water to remove the non adhering germs and the contamination level onto the surface is determined by direct imprint with Rodace agar plates.

Rodac® plate observation and reading are normally planned after 24 and 48 hours incubation at room temperature. Colony forming units (cfu) are counted with a microscope.

Culture duration and counting timing have to be adapted according the germ proliferation rate. Under normal conditions the final reading can be carried out 48 hours after Rodac® imprint. When germ proliferation is too slow, the final reading could be delayed up to 72 hours.

The results are expressed as a comparison of cfu between the treated tiles and the not-treated reference tiles. A prevention index can be defined as the ratio of the germ growth on treated tiles divided by the germ growth on not-treated tiles.

An extensive evaluation has been carried out on selected substrates. Many coated molecules were identified as delivering anti-germ adhesion properties on ceramic and/or on marble and/or stainless steel, but seldom on two or three surfaces at once. Only some nongermicial molecules were found effective and resistant to water rinse on more than one substrate.

Exceptional anti-germ adhesion effects were identified with various polysaccharides and with a glycoprotein (i.e. mucin). Especially effective and substantive (i.e. still effective after rinsing) on all tested substrates (i.e. ceramic, calcite and stainless steel), are Fucogel® 1000, xanthan gum, tragacanth and guar gums, as well as the glycoprotein mucin. Other tested gums are also effective but do not show the same versatility as the above; they are indeed effective, but with restrictions on specific substrate and/or on rinsing: karaya and gellan gums are effective and substantive on both ceramic and calcite substrates; carrageenan is effective on all tested substrates but is substantive on ceramic only; arabic gum is effective and substantive on ceramic and stainless steel only; polygalacturonic acid is moderately effective in some conditions.

Results are given in the following table expressed as prevention ratings for various coating molecules applied from 0.1% aqueous solutions (200 μl solution applied on 2.5×2.5 cm tile).

| | Prevention | | Prevention | | | |
|---|---|---|---|---|---|---|
| | rating | | rating | | Prevention rating | |
| | on ceramic | | on calcite | | on stainless steel | |
| Treatment | NR | R | NR | R | NR | R |
| Fucogel ® | 4 | 4 | 4 | 4 | 4 | 2 |
| Xanthan Gum | 4 | 4 | 4 | 4 | 2 | 1 |
| Iota Carrageenan | 3 | 3 | 4 | 0 | 3 | 0 |
| Gum Tragacanth | 4 | 4 | 4 | 4 | 4 | 2 |
| Gum Karaya | 3 | 4 | 4 | 4 | 4 | 0 |
| Polygalacturonic acid | 3 | 0 | 1 | 2 | 3 | 1 |
| Gum arabic | 3 | 3 | 2 | 0 | 3 | 3 |
| Mucin | 4 | 4 | 4 | 3 | 4 | 2 |
| Gelrite Gellan Gum | 3 | 4 | 4 | 2 | 4 | 0 |
| Guar Gum | 4 | 4 | 4 | 4 | 4 | 3 |

Prevention rating scale: 0 for 81 to 100% remaining germs (=no anti-adhesion effect); 1 for 61 to 80%; 2 for 41 to 60%; 3 for 21 to 40%; 4 for 1 to 20% and 5 for 0% (=no remaining germ). “NR” is for coatings not rinsed before inoculation while “R” is for coatings rinsed before inoculation (5×10 ml water/tile).

It was discovered that the very low amount of Fucogel® are needed to obtain a maximum efficacy. This fact is obvious at the reading of anti-germ adhesion ratings versus the Fucogel® concentration (see following table with same rating scale as above): too high a concentration is detrimental if the treatment is not rinsed. All rinsed treatments (even those with the lowest concentration) exhibit the highest observed performance (i.e. rating 4).

| | Prevention rating | | Prevention rating | |
|---|---|---|---|---|
| | on ceramic | | on calcite | |
| Treatment | NR | R | NR | R |
| 0.055% Fucogel ® | 4 | 4 | 3 | 4 |
| 0.11% Fucogel ® | 4 | 4 | 4 | 4 |
| 0.55% Fucogel ® | 3 | 4 | 2 | 4 |
| 1.1% Fucogel ® | 3 | 4 | 2 | 4 |

### Example 2

To assess whether surfactants have intrinsical anti-germ adhesion properties and whether the presence of surfactants would affect the activity of the anti-germ adhesion molecules already mentioned (i.e. polysaccharides or glycoprotein), a campaign of experiments has been carried out with using the same procedure as described in the above example 1 but with treating solutions made of single- or dual-surfactant compositions or made of anti-germ adhesion molecule admixed to single- or dual-surfactant compositions.

Results are given in the following table for treatment on three different substrates (ceramic, calcite and stainless steel 316L). The rating scale is identical as previously described. “NR” is for not rinsed coatings (before inoculation) while “R” is for rinsed coatings (5×10 ml water/tile).

In the following table the mentioned surfactants are: C9-C11 alkanol condensed with 8 moles of ethylene oxide (Neodol® 91-8); sodium lauryl sulfate (SLS); C12-13 alcohol EO 2:1 sodium sulfate (AEOS); cocoamidopropyl betaine (CAPB); C14-17 paraffin sodium sulfonate (PS); cocoamidopropyl amine oxide (CAPAO); C14-16 alpha olefin sodium sulfonate (AOS); lauryl polyglucose (APG); C10-14 linear alkylbenzenze sodium sulfonate (LAS); esterified polyethoxyether (Levenol®); soja lecithin (Lecithin).

| | Ceramic | | Calcite | | St. Steel | |
|---|---|---|---|---|---|---|
| | Prevention | | Prevention | | Pervention | |
| | Rating | | Rating | | Rating | |
| Treatment | NR | R | NR | R | NR | R |
| 2.5% Neodol ® 91-8/0.1% Fucogel ®/0.05% | 4 | 2 | 4 | 4 | — | — |
| perfume | | | | | | |
| 2.5% Neodol ® 91-8/0.1% Guar Gum/0.05% | 4 | 0 | 4 | 4 | — | — |
| perfume | | | | | | |
| 2.5% Neodol ® 91-8/0.1% Tragacanth/0.05% | 4 | 2 | 4 | 4 | — | — |
| perfume | | | | | | |
| 2.5% SLS/0.1% Fucogel ®/0.05% perfume | 4 | 4 | 4 | 4 | — | — |
| 2.5% SLS/0.1% Guar Gum/0.05% perfume | 4 | 4 | 4 | 4 | — | — |
| 2.5% SLS/0.1% Tragacanth/0.05% perfume | 4 | 5 | 4 | 4 | — | — |
| 2.5% SLS/0.11% Fucogel ® | 4 | 2 | 4 | 4 | — | — |
| 2.5% SLS/0.1% Guar Gum | 4 | 3 | 4 | 4 | — | — |
| 2.5% SLS/0.1% Tragacanth | 4 | 3 | 5 | 3 | — | — |
| 2.5% AEOS 2EO/0.1% Fucogel ® | 3 | 0 | 4 | 1 | — | — |
| 2.5% AEOS 2EO/0.1% Guar Gum | 4 | 2 | 4 | 4 | — | — |
| 2.5% AEOS 2EO/0.1% Tragacanth | 4 | 1 | 3 | 0 | — | — |
| 2.5% CAPB/0.1% Fucogel ® | 2 | 1 | 4 | 0 | — | — |
| 2.5% CAPB/0.1% Guar Gum | 2 | 0 | 2 | 0 | — | — |
| 2.5% CAPB/0.1% Tragacanth | 2 | 0 | 4 | 0 | — | — |
| 2.5% PS/0.1% Fucogel ® | 4 | 1 | 2 | 0 | — | — |
| 2.5% PS/0.1% Guar Gum | 4 | 2 | 4 | 3 | — | — |
| 2.5% PS/0.1% Tragacanth | 3 | 2 | 4 | 2 | — | — |
| 2.5% CAPAO/0.1% Fucogel ® | 3 | 2 | 4 | 3 | — | — |
| 2.5% CAPAO/0.1% Guar Gum | 2 | 0 | 3 | 1 | — | — |
| 2.5% CAPAO/0.1% Tragacanth | 3 | 0 | 4 | 1 | — | — |
| 2.5% AOS/0.1% Fucogel ® | 4 | 1 | 4 | 2 | — | — |
| 2.5% AOS/0.1% Guar Gum | 4 | 3 | 4 | 3 | — | — |
| 2.5% AOS/0.1% Tragacanth | 3 | 1 | 4 | 0 | — | — |
| 2.5% APG/0.1% Fucogel ® | 2 | 4 | 4 | 4 | — | — |
| 2.5% APG/0.1% Guar Gum | 0 | 0 | 2 | 1 | — | — |
| 2.5% APG/0.1% Tragacanth | 3 | 1 | 4 | 0 | — | — |
| 2.5% Lecithin/0.1% Fucogel ® | 0 | 0 | 4 | 3 | — | — |
| 2.5% Lecithin/0.1% Guar Gum | 0 | 2 | 4 | 4 | — | — |
| 2.5% Lecithin/0.1% Tragacanth | 0 | 3 | 4 | 3 | — | — |
| 2.5% Levenol ®/0.1% Fucogel ® | 2 | 0 | 0 | 2 | — | — |
| 2.5% Levenol ®/0.1% Guar gum | 3 | 0 | 3 | 1 | — | — |
| 2.5% Levenol ®/0.1% Tragacanth | 3 | 1 | 4 | 1 | — | — |
| 2.5% LAS/0.1% Fucogel ® | 4 | 4 | 4 | 4 | — | — |
| 2.5% LAS/0.1% Guar Gum | 4 | 4 | 4 | 4 | — | — |
| 2.5% LAS/0.1% Tragacanth | 4 | 4 | 4 | 4 | — | — |
| 2.5% LAS/0.1% Mucin | 4 | 4 | 4 | 4 | — | — |
| 1.25% LAS/1.25% Neodol ® 91-8/0.1% | 3 | 3 | 3 | 1 | 2 | 0 |
| Fucogel ® | | | | | | |
| 1.25% LAS/1.25% Neodol ® 91-8/0.1% Guar | 4 | 3 | 4 | 3 | 4 | 4 |
| Gum | | | | | | |
| 1.25% LAS/1.25% Neodol ® 91-8/0.1% | 4 | 1 | 3 | 2 | 4 | 1 |
| Tragacanth | | | | | | |
| 1.25% LAS/1.25% Neodol ® 91-8/0.1% Mucin | 4 | 2 | 3 | 2 | 3 | 2 |
| 1.25% LAS/1.25% Neodol ® 91-5/0.1% Guar | 4 | 3 | 4 | 4 | | |
| Gum | | | | | | |
| 1.25% LAS/1.25% APG/0.1% Fucogel ® | 4 | 4 | 4 | 1 | 4 | 0 |
| 1.25% LAS/1.25% APG/0.1% Guar Gum | 4 | 3 | 4 | 4 | 4 | 4 |
| 1.25% LAS/1.25% APG/0.1% Tragacanth | 4 | 3 | 4 | 2 | 4 | 2 |
| 1.25% LAS/1.25% APG/0.1% Mucin | 4 | 3 | 3 | 2 | 4 | 1 |
| 1.25% LAS/1.25% CAPB/0.1% Fucogel ® | 4 | 1 | 4 | 2 | — | — |
| 1.25% LAS/1.25% CAPB/0.1% Guar Gum | 4 | 0 | 3 | 3 | — | — |
| 1.25% LAS/1.25% CAPB/0.1% Tragacanth | 4 | 0 | 4 | 2 | — | — |
| 1.25% LAS/1.25% CAPB/0.1% Mucin | 4 | 0 | 4 | 1 | — | — |
| 1.25% LAS/1.25% Levenol ®/0.1% Fucogel ® | 2 | 0 | 4 | 2 | — | — |
| 1.25% LAS/1.25% Levenol ®/0.1% Guar | 4 | 0 | 4 | 3 | — | — |
| Gum | | | | | | |
| 1.25% LAS/1.25% Levenol ®/0.1% | 4 | 0 | 2 | 2 | — | — |
| Tragacanth | | | | | | |
| 1.25% LAS/1.25% Levenol ®/0.1% Mucin | 2 | 0 | 2 | 2 | — | — |
| 1.25% SLS/1.25% Neodol ® 91-8/0.1% | 3 | 1 | 4 | 0 | 4 | 0 |
| Fucogel ® | | | | | | |
| 1.25% SLS/1.25% Neodol ® 91-8/0.1% Guar | 4 | 2 | 4 | 1 | 4 | 2 |
| gum | | | | | | |
| 1.25% SLS/1.25% Neodol ® 91-8/0.1% | 4 | 2 | 4 | 3 | 4 | 1 |
| Tragacanth | | | | | | |
| 1.25% SLS/1.25% Neodol ® 91-8/0.1% Mucin | 3 | 0 | 4 | 0 | 4 | 1 |
| 1.25% SLS/1.25% APG/0.1% Fucogel ® | 4 | 0 | 4 | 3 | 4 | 2 |
| 1.25% SLS/1.25% APG/0.1% Guar gum | 4 | 0 | 4 | 2 | 4 | 0 |
| 1.25% SLS/1.25% APG/0.1% Tragacanth | 4 | 0 | 4 | 1 | 4 | 0 |
| 1.25% SLS/1.25% APG/0.1% Mucin | 4 | 0 | 4 | 1 | 4 | 0 |
| 1.25% SLS/1.25% CAPB/0.1% Fucogel ® | 4 | 1 | 4 | 2 | 3 | 2 |
| 1.25% SLS/1.25% CAPB/0.1% Guar gum | 4 | 1 | 4 | 3 | 3 | 0 |
| 1.25% SLS/1.25% CAPB/0.1% Tragacanth | 4 | 1 | 4 | 0 | 3 | 4 |
| 1.25% SLS/1.25% CAPB/0.1% Mucin | 2 | 1 | 4 | 3 | 2 | 0 |
| 1.25% SLS/1.25% Levenol ®/0.1% Fucogel ® | 4 | 0 | 5 | 2 | 3 | 2 |
| 1.25% SLS/1.25% Levenol ®/0.1% Guar | 4 | 0 | 5 | 2 | 3 | 1 |
| gum | | | | | | |
| 1.25% SLS/1.25% Levenol ®/0.1% | 4 | 0 | 5 | 1 | 4 | 1 |
| Tragacanth | | | | | | |
| 1.25% SLS/1.25% Levenol ®/0.1% Mucin | 3 | 0 | 4 | 0 | 3 | 0 |

It emerges from the above table that numerous single- or dual-surfactant compositions mixed with well selected polysaccharide or glycoprotein have the ability to impede or decrease germ adhesion on given substrates. Among the above mixed dual-surfactant compositions, the most effective ones in all tested conditions are 1.25% LAS/1.25% Neodol® 91-8/0.1% Guar Gum and 1.25% LAS/1.25% APG/0.1% Guar Gum.

What is claimed:

1. A nongermicidal composition which comprises approximately by weight:
(a) 0.025% to 3.0% of an organic polymer selected from the group consisting of polysaccharides, xanthan gum, tragacanth gum, guar gum, iota carrageenan, karaya gum, glycoprotein mucin and geirite gellan gum and mixtures thereof; and
(b) the balance being water.

2. A composition according to claim 1 further including at least one surfactant.

3. A composition of claim 2 further including a cosurfactant.

4. A composition according to claim 3 further including a perfume, essential oil and/or water insoluble.

5. A composition according to claim 4 further containing a polymeric thickener.

6. A composition according to claim 5 further including an inorganic builder salt.

7. A composition according to claim 2 further including a solubilizing agent.

8. A composition according to claim 7 further including a polymeric thickener.

9. A composition according to claim 2 further including an alkyl sulfate or a linear alkyl sulfonate or an alkyl polyglucoside or a lecithin.

10. A composition according to claim 2 further including more than one surfactant.

11. A composition according to claim 10 further including a linear alkyl benzene sulfonate and an alkyl polyglucoside.

12. A composition according to claim 10 further including a linear alkyl benzene sulfonate and an ethoxylated alcohol.

13. A composition according to claim 10 further including guar gum.
